(19) United States
(12) Patent Application Publication
(10) Pub. No.: US 2005/0255483 A1
(43) Pub. Date: Nov. 17, 2005

(54) System and method for smoothing melting curve data

(75) Inventors: Alan Ridgeway Orr, San Diego, CA (US); Roger Harry Taylor, San Diego, CA (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 10/852,788

(22) Filed: May 25, 2004

Related U.S. Application Data
(60) Provisional application No. 60/571,404, filed on May 14, 2004.

(51) Int. Cl. C12Q 1/68; G06F 19/00; G01N 33/48; G01N 33/50

(52) U.S. Cl. 435/6; 702/20

(57) ABSTRACT

The present invention is a system and method for smoothing melting curve data to help identify a specific sequence of double stranded DNA sample after performing a polymerase chain reaction. The system comprises a plurality of measurements of a fluorescence and a temperature of a dye over a temperature range to create a raw array; calculating a collapse number using a temperature resolution that facilitates the smoothing of the melting curve data, a number of data points in the raw array, and a temperature data range from the raw array; calculating a plurality of fluorescence averages and a plurality of temperature averages from the collapse number and the raw array; and generating a smooth array from the plurality of fluorescence averages and the plurality of temperature averages wherein the smooth array decreases a variability in the raw array.

### RELATED APPLICATIONS

This application claims the benefit of U.S. Provisional Application No. 60/571,404 filed on May 14, 2004. The entire teachings of the above application is incorporated herein by reference.

### FIELD OF THE INVENTION

The present invention relates to identifying a specific sequence of double stranded DNA in a sample after performing polymerase chain reaction, and more particularly to a system and a method for smoothing melting curve data.

### BACKGROUND OF INVENTION

The identification of a specific sequence of double stranded DNA (dsDNA) in a sample after performing a polymerase chain reaction (PCR) is often difficult. Many variables including the test conditions, sample size and the frequency of measurements affect the often low signal to noise ratio that makes data analysis difficult. Improvements to the signal to noise ratio are dependent on utilizing and manipulating data generated during the test period.

PCR is well known in the art and generally refers to an in vitro method for amplifying a specific polynucleotide template sequence. The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference.

The use of a dye that binds with dsDNA can facilitate the identification of dsDNA in a sample because some dyes increase in fluorescence when bound to dsDNA. SYBR Green I is a well known example of a dye with this property used to identify dsDNA in a sample. The fluorescence signal of such a dye is proportional to the total quantity of dsDNA in a sample. The use of such a dye with a melting curve enhances the ability to identify a specific sequence of dsDNA in a sample.

A melting curve is a graphical tool used in the detection of a specific sequence of dsDNA. The melting curve measures the fluorescence of the specific sample as a function of temperature. The melting temperature of a dsDNA sequence is the temperature at which half of the dsDNA sequence dissociates into single stranded DNA. Dissociation, or denaturation, is a process by which the individual strands of the dsDNA separate into single stranded DNA (ssDNA). The combined effect that different dsDNA sequences often have different melting temperatures and the fluorescence of the dye decreases when not bound to dsDNA allows the fluorescence signal from a melting curve to be used to help determine the dsDNA sequence found in a post PCR sample.

An apparatus for thermally cycling samples of a biological material that produces melting curve data is disclosed in assignee's U.S. Pat. No. 6,657,169 and U.S. patent application Ser. No. 10/691,874, the entirety of the patent and patent application are hereby incorporated by reference. In addition, a flexible heating cover assembly for thermal cycling samples of a biological material is disclosed in U.S. Pat. No. 6,730,883 and U.S. patent application Ser. No. 10/811,663, the entirety of the patent and patent application are hereby incorporated by reference.

To best use a melting curve to determine the melting temperatures of different dsDNA in a sample, the derivative of the fluorescence versus temperature plot is utilized. However, the mathematical process of producing a derivative plot effectively reduces the signal level without substantially changing the noise level. The resulting reduction in the signal to noise ratio of the melting curve derivative presents a problem for subsequent data analysis and decision making processes. Noise in the temperature measurement introduces additional noise into the melting curve, further complicating the data analysis.

One method to increase the signal to noise ratio is to smooth the melting curve data. Smoothing melting curve data improves the signal to noise ratio, facilitating the data analysis and the detection of a specific sequence of dsDNA. By smoothing the melting curve data, the noise in the unsmoothed melting curve data is reduced, thereby facilitating subsequent data analysis and decision making processes.

U.S. Pat. No. 6,711,515 to Lehtinen et al. discloses a method for smoothing measurement results using a parameter comprising weight factors from a number of measurements in a sample. The Lehtinen et al. method does not require a large sample in order to not react to individual deviating measurement results. The Lehtinen et al. method uses a measure of a center which has a high breaking point and which for the calculation takes into account the reliability or importance of the measurement points with the aid of weight factors.

U.S. Pat. No. 6,664,064 to Dietmaier discloses a method for melting curve analysis of repetitive PCR products. The Dietmaier method correlates the melting point temperature with the number of repeats present in a target nucleic acid. However, Dietmaier does not disclose methods to reduce the noise of melting curve data or to smooth the melting curve data.

U.S. Pat. No. 6,506,568 to Shriver et al. discloses a method of analyzing single nucleotide polymorphisms using melting curve and restriction endonuclease digestion. The Shriver et al. method comprises the steps of DNA amplification, restriction enzyme digestion and melting curve analysis. The Shriver et al. method does not provide a procedure to reduce the high variability in the melting curve data to reduce the noise in the data.

The prior art does not provide a solution for efficiently and accurately smoothing melting curve data to allow for detection of a specific sequence of dsDNA. The prior art does not provide a solution for managing data that deviates considerably from other data values in the melting curve data set. There remains a need in the art for a method of reducing the variability in the melting curve data to allow for detection of a specific sequence of dsDNA. There also remains a need in the art for a system and method for smoothing melting curve data that is easy to use, accurate, and facilitates data analysis of melting curve data.

### SUMMARY OF THE INVENTION

The present invention is a system of smoothing melting curve data comprising: measuring a fluorescence and a temperature of a dye over a temperature range to create a raw array; calculating a collapse number using a temperature resolution that facilitates the smoothing of the melting curve data, a number of data points in the raw array, and a temperature data range from the raw array; calculating a plurality of fluorescence averages and a plurality of temperature averages from the collapse number and the raw array; and generating a smooth array from the plurality of fluorescence averages and the plurality of temperature averages wherein the smooth array decreases a variability in the raw array.

The present invention is a system for decreasing the variability in melting curve data comprising: means for measuring a plurality of fluorescence values and a plurality of temperature values over a temperature range to create a raw array; means for calculating a collapse number from the raw array and a temperature resolution; means for averaging the plurality of fluorescence values and the plurality of temperature values using the collapse number; and means for generating a smooth array from the plurality of fluorescence averages and the plurality of temperature averages.

The present invention is also a method of increasing a signal to noise ratio of melting curve data. A fluorescence of a dye at a temperature is measured over a plurality of temperature increments of a temperature range to create a raw array. A collapse number is calculated using a temperature resolution that facilitates increasing the signal to noise ratio of the melting curve data, a number of data points in the raw array, and a temperature data range of the raw array. A plurality of fluorescence averages and a plurality of temperature averages are calculated from the collapse number and the raw array, and a smooth array is generated from the plurality of fluorescence averages and the plurality of temperature averages. A derivative array is generated from the smooth array.

The present invention is also a melting curve smoothing algorithm comprising a means for measuring a plurality of fluorescence values and a plurality of temperature values. The melting curve smoothing algorithm also comprises a means for generating a smooth array by calculating a collapse number based on the raw array and a specified temperature resolution and averaging the plurality of fluorescence values and the plurality of temperature values using the collapse number.

The present invention is a method of decreasing a variability in melting curve data. The fluorescence and temperature of a dye is measured over a plurality of temperature increments of a temperature range to create a new array. A collapse number is calculated based on a number of data points in the raw array, a temperature data range of the raw array, and a temperature resolution that decreases a variability in the raw array. A plurality of fluorescence averages and a plurality of temperature averages are calculated from the collapse number and the raw array, a smooth array is generated from the plurality of fluorescence averages and the plurality of temperature averages, and a derivative array is generated from the smooth array. The presence of a specific sequence of dsDNA in a sample after PCR is determined.

The present invention is a computer readable medium containing program instructions for smoothing melting curve data comprising: program instructions for measuring the fluorescence and temperature of a dye over a temperature range to create a raw array; program instructions for calculating a collapse number using a temperature resolution that facilitates the smoothing of the melting curve data, a number of data points in the raw array, and a temperature data range from the raw array; program instructions for calculating a plurality of fluorescence averages and a plurality of temperature averages from the collapse number and the raw array; program instructions generating a smooth array from the plurality of fluorescence averages and the plurality of temperature averages; and program instructions for generating a derivative array from the smooth array.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a schematic diagram showing an overview of a melting curve variable resolution smoothing algorithm of the present invention.

FIG. 2 is a block diagram of a collapse number calculation for a melting curve variable resolution smoothing algorithm of the present invention.

FIG. 3 is a block diagram for providing a smoothed melting curve of a melting curve variable resolution smoothing algorithm of the present invention.

FIG. 4 is a melting curve showing a raw array (solid lines) and a smooth array (dashed lines) generated from a melting curve variable resolution smoothing algorithm of the present invention.

FIG. 5 is a derivative of the melting curve showing a derivative of a raw array (solid lines) and a derivative of a smooth array (dashed lines) generated from a melting curve variable resolution smoothing algorithm of the present invention.

FIG. 6 is a block diagram of a computer system for carrying out a smoothing of melting curve data according to the present invention.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides a system and method for smoothing melting curve data for identification of a specific sequence of DNA sample after performing a polymerase chain reaction. A fluorescence and a temperature of a dye is measured over a temperature range to create a raw array, and a smooth array is generated from a plurality of fluorescence averages and a plurality of temperature averages of the raw array. The smooth array decreases a variability in the raw array and increases a signal to noise ratio of the melting curve data.

The following terms and definitions are used herein:

“Melting Curve” as used herein refers to a graphical tool representing a fluorescence of a sample as a function of temperature.

“Raw Array” as used herein refers to the data of the fluorescence versus temperature of a sample.

“Smooth Array” as used herein refers to data generated from a melting curve variable resolution smoothing algorithm applied to a raw array.

“Derivative Array” as used herein refers to data representing the rate of change of another data set.

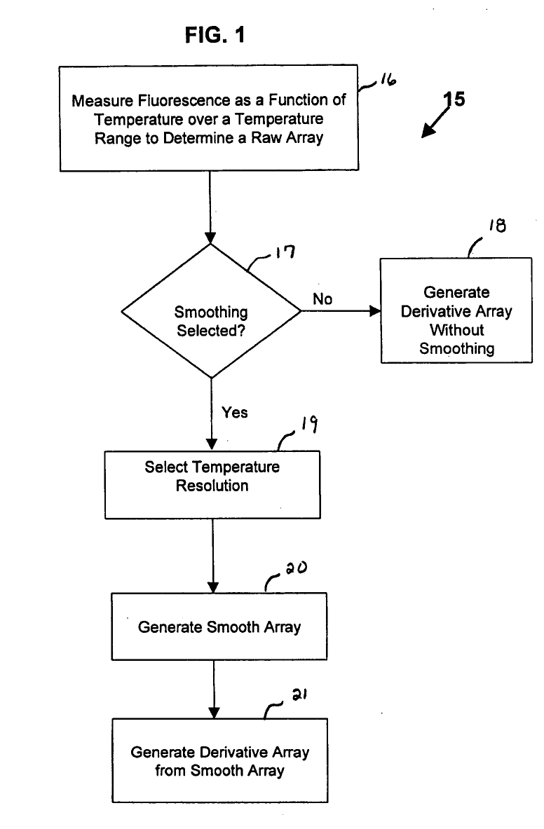

A schematic diagram of an overview of a melting curve variable resolution smoothing algorithm (MCVRSA) of the present invention is shown generally at 15 in FIG. 1. During a melting curve test procedure, a melting curve is generated by measuring a fluorescence and a temperature of a dye at various temperatures over a temperature range 16. In a preferred embodiment of the present invention, a single fluorescence is measured at each temperature over the temperature range. In another embodiment of the present invention, a plurality of fluorescence measurements are taken at each temperature over the temperature range. The data of the fluorescence versus temperature is a raw array.

Melting curve data can be obtained from samples containing appropriate fluorescent moities processed by any instrument or method for conducting thermal cycling, PCR, quantitative PCR or similar processing. Melting curve data can be obtained from any fluorometric or spectrophotometric apparatus equipped with a means of adjusting the sample temperature to above the melting temperature of the DNA sample. Examples of such instruments include, but are not limited to, thermal cyclers (both modular and multi-block), optical thermocyclers commonly used for quantitative PCR, fluorometers with temperature control, PCR machines, batch heaters or chillers, and other similar instruments, all of which are equipped with associated optics so as to permit generation and maintenance of specific temperatures for a defined period of time while measuring fluorescence. Those skilled in the art will recognize other instruments or methods known in the art used in connection with the generation of melting curve data are within the spirit and scope of the present invention.

In a preferred embodiment of the present invention, DNA is heated in the presence of a dye during a melting curve test procedure. In an alternative embodiment of the present invention, DNA is heated in the presence of a probe during a melting curve test procedure. In a typical melting curve test procedure, the DNA sample, in the presence of the dye, is heated to an initial temperature, and the fluorescence at the initial temperature is measured. The test temperature is increased over a series of temperature increments to a final test temperature. In another embodiment of the present invention, the test temperature can be increased or decreased over a series of temperature increments to create the raw array. The fluorescence and the temperature of the dye are measured at each of the temperatures of each temperature increment. In one embodiment of the present invention, the temperature increments are constant. In another embodiment of the present invention, the temperature increments are not constant.

The preferred dye used with the DNA sample is SYBR Green I. In another embodiment of the present invention, the dye is SYBR Green II, ethidium bromide or other dye that binds to ssDNA or dsDNA. Those skilled in the art will recognize that other dyes known in the art can be used with DNA to generate melting curves that are within the spirit and scope of the present invention.

After generation of the melting curve with the raw array, a smoothing option 17 is selected. If smoothing is not selected, a derivative array is generated from the raw array 18. In a preferred embodiment of the present invention, the derivative array is determined by mathematical calculations of the difference of the fluorescence measurements of sequential pairs of points in the raw array and the average of the temperature of these pairs of sequential points. Those skilled in the art will recognize the derivative array can be generated from many methods known in the art and still be within the spirit and scope of the present invention. The derivative array uses the raw array to determine the melting temperatures of different dsDNA in a sample to facilitate detection of a specific sequence of dsDNA.

If smoothing is selected, a temperature resolution that is greater than the temperature increments used to record the raw array is chosen 19. The temperature resolution is chosen to decrease the variability of the fluorescence and temperature measurements and is based in part on the temperature increments and a number of data points in the raw array. Generally, an increase in the temperature resolution increases a signal to noise ratio to facilitate determination of the specific sequence of the dsDNA. However, in some cases, for example, if the melting temperatures of different sequences of dsDNA differ by less than the temperature resolution, an increase in the temperature resolution reduces the ability to differentiate between specific sequences of the dsDNA.

Noise refers to a variability in a measurement that introduces an amount of error in subsequent calculations to decrease the ability to determine specific sequences of dsDNA in a sample after PCR. Noise can be characterized by sharp increases or decreases in a measurement from a previous measurement. The presence of noise results from a plurality of factors including, but not limited to, technical and mechanical limitations of the measurement system test conditions, and other factors known to those skilled in the art.

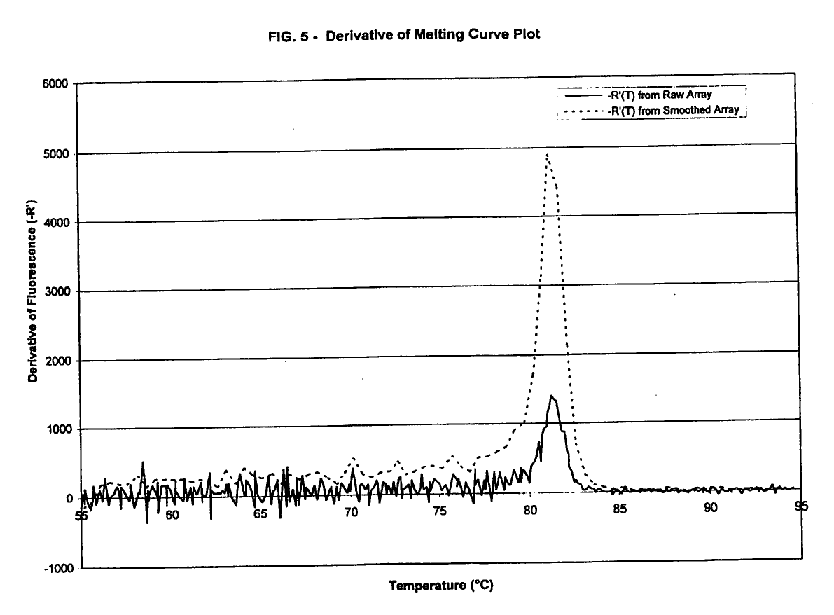

The signal to noise ratio represents a measure of the variability in the melting curve data and the ability to utilize the melting curve and a derivative of the melting curve to detect a specific sequence of dsDNA. As described in more detail in FIG. 5, the MCVRSA of the present invention produces a more distinct plot of the derivative of the melting curve (from the smooth array) with a more distinct fluorescence peak, or higher signal, when compared to the plot of the derivative of the melting curve with the raw array. The higher signal and reduced noise from the MCVRSA of the present invention facilitates the detection of a specific sequence of dsDNA.

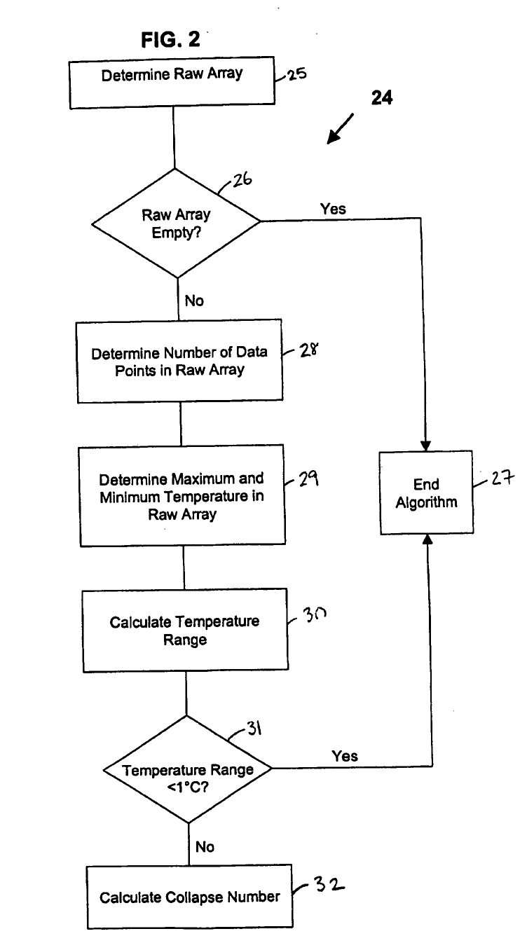

With the smoothing selection chosen, a smooth array 20 (further explained and detailed in FIG. 2) is generated. After generation of the smooth array 20, in a preferred embodiment of the present invention, a derivative array is generated 21 by calculating the difference of the fluorescence measurements of sequential pairs of points in the smooth array and the average of the temperatures of these pairs of points.

FIG. 2 shows a block diagram 24 for the calculation of a collapse number 32 for the MCVRSA. With the raw array as input to the MCVRSA 25, an error checking step 26 that is unrelated to the MCVRSA is run based upon the content of the raw array. If the raw array is empty, the MCVRSA algorithm is ended 27. If the raw array is not empty, the number of data points in the raw array is counted 28. Both the maximum temperature in the raw array and the minimum temperature in the raw array are determined 29 and a “temperature range” is calculated 30 by subtracting the minimum temperature from the maximum temperature. Equation 1 illustrates the calculation for the temperature range.
Temperature Range=Temperatureₘₐₓₘᵤₘ−Temperatureₘᵢₙᵢₘᵤₘ (1)
An additional error checking step 31 is run based upon the temperature range. If the temperature range is less than 1° Celsius (C), the algorithm is ended 27. If the temperature range is greater than 1° C., a “collapse number” is calculated 32. The calculation for the collapse number is given in Equation 2.

$$Collapse Number = max ( 1 , \frac{N * W}{Temperature Range} ) \qquad ( 2 )$$

where max( ) is a function that returns the maximum value in the parentheses, N is the number of data points in the raw array, W is the temperature resolution, and Temperature Range is the difference between the maximum and minimum temperature as given by Equation 1.

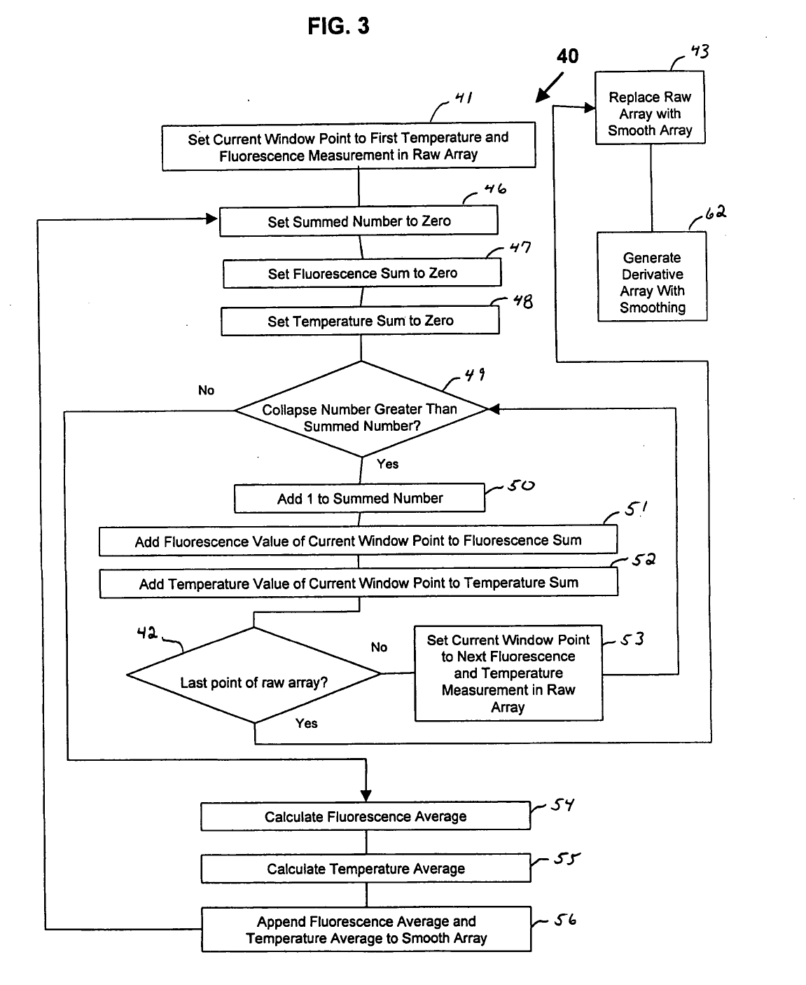

FIG. 3 is a block diagram 40 for providing a smoothed melting curve of the MCVRSA of the present invention. FIG. 3 indicates steps for generating a smooth array ending in the creation of a derivative array from the smooth array. FIG. 3 uses the raw array, the number of data points in the raw array (N), the temperature resolution (W), the temperature range and the collapse number to determine the smooth array. As shown in FIG. 3, the MCVRSA comprises a plurality of looping steps to generate the smooth array.

Initially, the first temperature and fluorescence measurement in the raw array is set to a current window point 41. The current window point is a representative name within the looping steps of the MCVRSA for specific sequential fluorescence and temperature measurements of the raw array. A loop counter, hereinafter referred to as “summed number,” is used for the data points in a window corresponding to the collapse number. The summed number is initially set to a value of zero (0) 46. A parameter known as a fluorescence sum, representing a summation of a group of fluorescence values in a window of the raw array corresponding to the collapse number, is initially set to zero (0) 47. A parameter known as a temperature sum, representing a summation of a group of temperature values in a window of the raw array, is also initially set to zero (0) 48.

A window summing loop is executed to calculate the fluorescence sum and the temperature sum in the window as long as the collapse number is greater than the summed number. The window corresponds to a portion of the data points in the raw array and is a function of the collapse number and the summed number. If the collapse number is greater than the summed number 49, the window summing loop continues until the collapse number is less than or equal to the summed number. Within the window summing loop, the summed number is incremented by one (1) 50 and the fluorescence value of the current window point is added to the fluorescence sum 51. In the same manner, the temperature value of the current window point is added to the temperature sum 52.

If the current window point is the last point of the raw array 42, the raw array is replaced with the smooth array 43. From the smooth array, the derivative array is generated 62. The calculations of the derivative array are described in the description of FIG. 1. As will be illustrated for the subsequent steps of the block diagram of FIG. 3, the smooth array is a plurality of fluorescence averages and a plurality of temperature averages. If the current window point is not the last point of the raw array, a series of calculations are performed to generate the smooth array.

Next, the current window point is set to the next fluorescence and temperature measurement in the raw array 53 of the window. The window summing loop continues as long as the collapse number is greater than the summed number 49. Within the window summing loop, a fluorescence sum and a temperature sum are generated.

Once the collapse number is less than or equal to the summed number, the fluorescence average 54 and the temperature average 55 are calculated for the specific window. The fluorescence average and the temperature average are appended into the smooth array 56 to generate smoothed melting curve data. Equation 3 gives the calculation for the fluorescence average.

$$Fluorescence Average = \frac{1}{CollapseNumber} {\sum }_{i = 1}^{CollapseNumber} {fluorescence}_{i} \qquad ( 3 )$$

where (Σ fluorescence) is the fluorescence sum described in the window summing loop. Equation 4 gives the calculation for the temperature average.

$$Temperature Average = \frac{1}{CollapseNumber} {\sum }_{i = 1}^{CollapseNumber} {temperature}_{i} \qquad ( 4 )$$

where (Σ temperature) is the temperature sum described in the window summing loop.

The entire looping procedure is repeated until all of the fluorescence and temperature points in the raw array have been used in the window summing loop. Since the MCVRSA is a sequential, non-overlapping algorithm, fluorescence and temperature measurements of the raw array are used at most once in the calculations. In other words, no additional windows can be generated after all of the fluorescence and temperature measurements have gone through the window summing loop

The MCVRSA reduces the variability in the temperature and fluorescence measurements of the raw array by calculating the fluorescence average and the temperature average in generating the smooth array. To best use the melting curve to determine the melting temperatures of different dsDNA that may exist in a DNA sample, the derivative of the fluorescence versus temperature plot is used. The data points of the derivative of the fluorescence versus temperature plot are the derivative array. However, without smoothing the melting curve data to produce the smooth array, the signal to noise ratio is often too low to distinguish the melting temperature of different dsDNA.

Noise in the melting curve derivative is often a problem because the mathematical process of taking a derivative produces a signal that is smaller than the fluorescence signal but does not substantially change the noise level. Noise in the temperature measurement also introduces noise into the melting curve. Smoothing the melting curve data mitigates the reduction in the signal to noise ratio. As a result, the smoothing of the melting curve data reduces the noise in the melting curve derivative, allowing for better discrimination between specific sequences of dsDNA in a sample after completion of PCR.

The MCVRSA of the present invention is most effective when the fluorescence is measured at a temperature resolution tighter than is required to resolve the different melting temperatures of different sequences of dsDNA and then to smooth the fluorescence and temperature measurements over a range of temperatures that provides adequate temperature resolution. The MCVRSA of the present invention improves the signal to noise ratio by increasing the separation between measurements as well as averaging multiple fluorescence values. Averaging over multiple values decreases the noise, thereby increasing the signal to noise ratio. The signal to noise ratio of the derivative array is also increased because the MCVRSA increases its signal by creating larger temperature separations for the calculation of the derivative of the temperature and fluorescence points. Larger temperature separations correspond to larger differences in signal, and larger differences in signal mean larger slopes, which is the parameter represented in the derivative array. This improvement in signal may not be significant in the smooth array, but it can be significant in the derivative array.

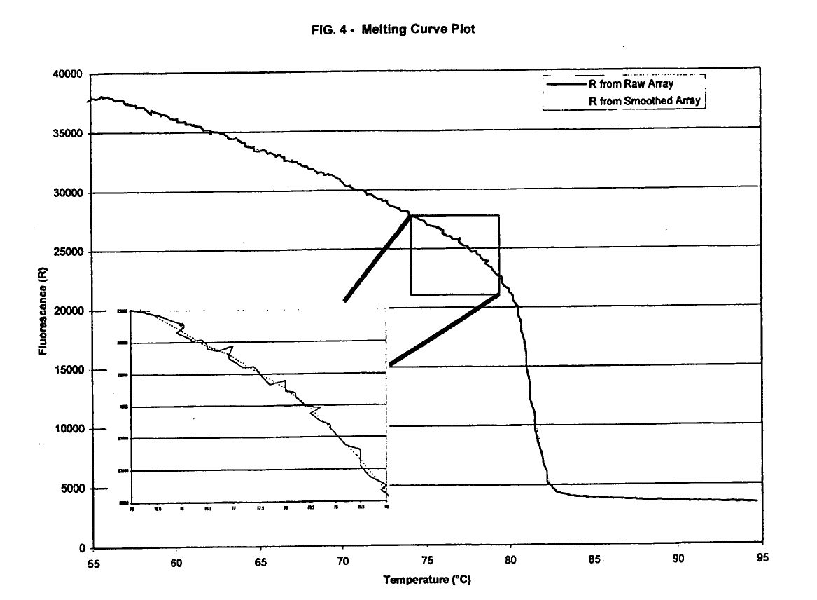

FIG. 4 shows a representative melting curve with both the raw array and the smooth array. FIG. 4 also shows an exploded view of a portion of the melting curve to show the difference in the variability between the raw array and the smooth array. The solid line in FIG. 4 represents the raw array and the dashed line represents the smooth array. The vertical Y axis represents the fluorescence, R, and the horizontal X axis represents the test temperatures. The variability in the fluorescence and the temperature is reduced with the MCVRSA to produce the smooth array that eliminates the sharp jumps in the fluorescence measurements of the raw array. A more pronounced effect of the smoothing of the melting curve data with the MCVRSA is shown in the derivative of the melting curve.

FIG. 5 shows a representative derivative of the melting curve with both the derivative of the raw array and the derivative of the smooth array. The solid line in FIG. 5 represents the derivative of the raw array and the dashed line represents the derivative of the smooth array. The vertical Y axis represents the negative derivative of the fluorescence, ⁻R′, and the horizontal X axis represents the temperature. As shown in FIG. 5, the variability in the derivative of the fluorescence and the temperature is reduced with the MCVRSA to produce the smooth array. Most notably, the derivative of the smooth array gives a significantly higher signal as shown by the peak in the derivative of the fluorescence. The signal of the derivative of the smooth array is approximately four times higher than the derivative of the raw array at this peak. By decreasing the noise and increasing the signal, the derivative of the smooth array plot facilitates determination of a specific sequence of dsDNA in a sample after PCR.

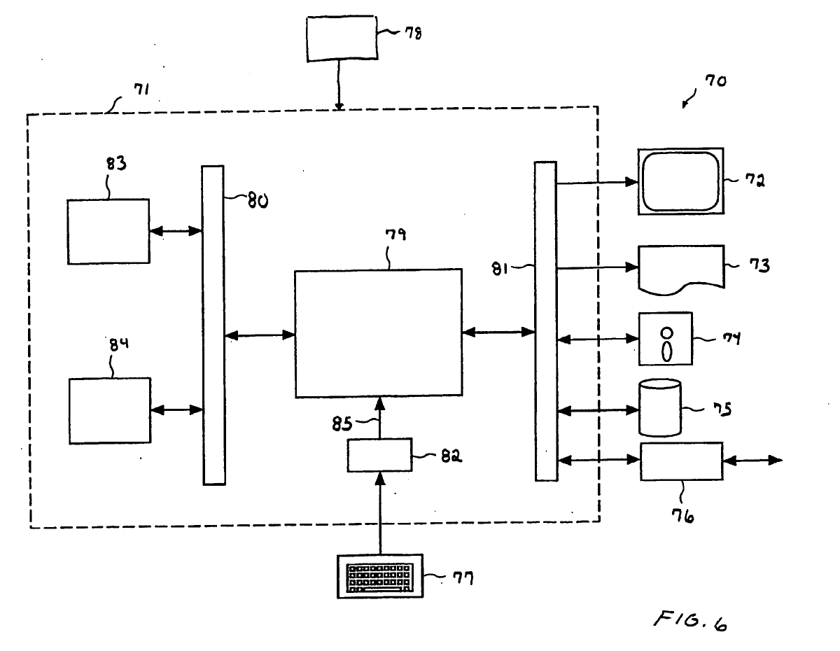

FIG. 6 shows a block diagram of a computer system 70 for carrying out the MCVRSA of the present invention. In another embodiment of the present invention, the computer system is an embedded computer system. The computer system 70 includes a computer 71, a display screen (or monitor) 72, a printer 73, a floppy disk drive 74, a hard disk drive 75, a network interface 76 and a keyboard 77. The raw array from the PCR test 78 is input into the computer 71. The computer 71 includes a microprocessor 79, a memory bus 80, a peripheral bus 81 and a keyboard controller 82. The keyboard 77 is used by a user to input commands and other instructions to the computer system 70. The keyboard controller 82 receives input from the keyboard 77 and sends decoded symbols for each pressed key of the keyboard 77 to the microprocessor 79 over a bus 85. The computer 71 can be a personal computer, a workstation or some other type of computer.

The microprocessor 79 controls the operation of the computer system 70. The microprocessor 79 uses instructions received from memory to control the reception and manipulation of the raw array 78 through the MCVRSA and outputs and displays the data on output devices. The keyboard 77 is used by a user to input commands and other instructions to the computer system 70. The memory bus 80 is used by the microprocessor 79 to access a random access memory (RAM) 83 and a read only memory (ROM) 84. The RAM 83 is used by the microprocessor 79 as a general storage area and the ROM 84 is used to store the instructions or program code of the MCVRSA followed by the microprocessor 79. The computer code and data may reside on the RAM 83, the ROM 84, the hard disk drive 75 or a removable program medium that can be loaded or installed on the computer system 70.

The peripheral bus 81 is used to access the input, output and storage devices used by the computer 71. These input, output and storage devices include, but are not limited to, the display screen 72 the printer 73, the floppy disk drive 74, the hard disk drive 75 and the network interface 76.

The display screen 72 displays images of the melting curve data provided by the microprocessor 79 via the peripheral bus 81 or provided by other components in the computer system 70. The printer 73 provides an image on a sheet of paper or a similar type of surface. Other output devices including, but not limited to, plotters or typesetters can be used in place of, or in addition to, the printer 73.

The floppy disk drive 74 and the hard disk drive 75 are used to store various types of data. The floppy disk drive 74 facilitates transporting the data to a separate computer system while the hard disk drive 75 allows fast access to large amounts of stored data. The network interface 76 is used to send and receive data over a network that is connected to other computer systems. Those skilled in the art will recognize that other persistant storage devices to store various types of data are known in the art and are within the spirit and scope of the present invention.

The MCVRSA of the present invention provides several benefits by smoothing the melting curve data. The MCVRSA of the present invention improves the signal to noise ratio of the melting curve data and reduces the variability in the raw array. The MCVRSA provides the smooth array to facilitate determination of a specific sequence of dsDNA. The MCVRSA of the present invention provides a system and a method for smoothing melting curve data that is easy to use, accurate and facilitates data analysis of melting curve data.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A system of smoothing melting curve data comprising:
measuring a fluorescence and a temperature of a dye over a temperature range to create a raw array;
calculating a collapse number using a temperature resolution that facilitates the smoothing of the melting curve data, a number of data points in the raw array, and a temperature data range from the raw array;
calculating a plurality of fluorescence averages and a plurality of temperature averages from the collapse number and the raw array; and
generating a smooth array from the plurality of fluorescence averages and the plurality of temperature averages,
wherein the smooth array decreases a variability in the raw array.

2. The system of claim 1 further comprising generating a derivative array from the smooth array.

3. The system of claim 1 wherein the decrease in variability in the raw array facilitates the identification of a specific sequence of double stranded DNA in a sample after polymerase chain reaction.

4. The system of claim 1 further comprising measuring the fluorescence and the temperature of the dye at each temperature over a plurality of temperature increments of the temperature range.

5. The system of claim 1 further comprising determining a number of data points in the raw array.

6. The system of claim 1 further comprising calculating the temperature data range from a difference between a maximum temperature and a minimum temperature of the temperature range.

7. The system of claim 1 further comprising generating the plurality of fluorescence averages and the plurality of temperature averages in a window summing loop by comparing the collapse number and a summed number.

8. The system of claim 7 wherein the summed number is a loop counter.

9. The system of claim 7 wherein the fluorescence average and the temperature average are calculated if the collapse number is less than the summed number.

10. A system for decreasing the variability in melting curve data comprising:
means for measuring a plurality of fluorescence values and a plurality of temperature values over a temperature range to create a raw array;
means for calculating a collapse number from the raw array;
means for averaging the plurality of fluorescence values and the plurality of temperature values using the collapse number; and
means for generating a smooth array from the plurality of fluorescence averages and the plurality of temperature averages.

11. The system of claim 10 wherein the means of measuring a plurality of fluorescence values and a plurality of temperature values comprises measuring a fluorescence at a temperature over a series of temperature increments of the temperature range.

12. The system of claim 10 wherein the means of calculating the collapse number comprises determining a number of data points in the raw array.

13. The system of claim 10 wherein the means of calculating the collapse number comprises choosing a temperature resolution to decrease the variability in melting curve data.

14. The system of claim 10 wherein the means of calculating the collapse number comprises determining a temperature data range.

15. The system of claim 10 further comprising means for generating a plurality of fluorescence averages and a plurality of temperature averages in a loop by comparing the collapse number and a summed number.

16. The system of claim 10 further comprising means for generating a derivative array from the smooth array.

17. A method of increasing a signal to noise ratio of melting curve data comprising:
measuring a fluorescence and a temperature of a dye over a plurality of temperature increments of a temperature range to create a raw array;
calculating a collapse number using a temperature resolution that facilitates increasing the signal to noise ratio of the melting curve data, a number of data points in the raw array, and a temperature data range from the raw array;
calculating a plurality of fluorescence averages and a plurality of temperature averages from the collapse number and the raw array; and
generating a smooth array from the plurality of fluorescence averages and the plurality of temperature averages; and
generating a derivative array from the smooth array.

18. The method of claim 17 wherein the smooth array decreases a variability in the raw array.

19. The method of claim 18 wherein the decrease in variability of the raw array facilitates an identification of a specific sequence of double stranded DNA in a sample after polymerase chain reaction.

20. The method of claim 17 further comprising determining a number of data points in the raw array.

21. The method of claim 17 further comprising calculating the temperature range from a difference between a maximum temperature and a minimum temperature of the raw array.

22. The method of claim 17 further comprising generating the plurality of fluorescence averages and the plurality of temperature averages in a window summing loop by comparing the collapse number and a summed number.

23. The method of claim 22 wherein the summed number is a loop counter.

24. The method of claim 22 wherein a fluorescence average and a temperature average are calculated if the collapse number is less than the summed number.

25. A melting curve smoothing algorithm comprising:
means for measuring a plurality of fluorescence values and a plurality of temperature values over a temperature range to create a raw array;
means for generating a smooth array, the means for generating the smooth array comprising;
means for calculating a collapse number from the raw array; and
means for averaging the plurality of fluorescence values and the plurality of temperature values using the collapse number.

26. The melting curve smoothing algorithm of claim 25 wherein the means of calculating the collapse number comprises determining a number of data points in the raw array.

27. The melting curve smoothing algorithm of claim 25 wherein the means of calculating the collapse number comprises choosing a temperature resolution to facilitate smoothing of the melting curve.

28. The melting curve smoothing algorithm of claim 25 wherein the means of calculating the collapse number comprises determining a temperature data range.

29. The melting curve smoothing algorithm of claim 25 further comprising means for generating a plurality of fluorescence averages and a plurality of temperature averages in a window summing loop by comparing the collapse number and a summed number.

30. A method of decreasing a variability in melting curve data comprising:
measuring a fluorescence and a temperature of a dye over a plurality of temperature increments of a temperature range to create a raw array;
calculating a collapse number using a temperature resolution that decreases a variability in the raw array, a number of data points in the raw array, and a temperature data range from the raw array;
calculating a plurality of fluorescence averages and a plurality of temperature averages from the collapse number and the raw array;
generating a smooth array from the plurality of fluorescence averages and the plurality of temperature averages;
generating a derivative array from the smooth array; and
determining the presence of a specific sequence of double stranded DNA in a sample after polymerase chain reaction.

31. The method of claim 30 further comprising determining a number of data points in the raw array.

32. The method of claim 30 further comprising calculating the temperature data range from a difference between a maximum temperature and a minimum temperature of the raw array.

33. The method of claim 30 further comprising generating the plurality of fluorescence averages and the plurality of temperature averages in a window summing loop by comparing the collapse number and a summed number.

34. The method of claim 33 wherein a fluorescence average and a temperature average are calculated if the collapse number is less than a summed number.

35. A computer readable medium containing program instructions for smoothing melting curve data comprising:
program instructions for measuring a fluorescence and a temperature of a dye over a temperature range to create a raw array;
program instructions for calculating a collapse number using a temperature resolution that facilitates the smoothing of the melting curve data, a number of data points in the raw array, and a temperature data range from the raw array;
program instructions for calculating a plurality of fluorescence averages and a plurality of temperature averages from the collapse number and the raw array;
program instructions generating a smooth array from the plurality of fluorescence averages and the plurality of temperature averages; and
program instructions for generating a derivative array from the smooth array.

36. The computer readable medium of claim 35 further comprising program instructions for determining a number of data points in the raw array.

37. The computer readable medium of claim 35 further comprising program instructions for calculating the temperature data range from a difference between a maximum temperature and a minimum temperature.
